(19) United States
(12) Patent Application Publication
(10) Pub. No.: US 2025/0195857 A1
(43) Pub. Date: Jun. 19, 2025

(54) SOLID DRUG RELEASING BIOABSORBABLE PROSTATE IMPLANT FOR BENIGN PROSTATIC HYPERPLASIA

(71) Applicant: Resurge Therapeutics Inc., San Jose, CA (US)

(72) Inventors: Mikael Trollsas, San Jose, CA (US); John Stankus, San Jose, CA (US)

(21) Appl. No.: 18/545,564

(22) Filed: Dec. 19, 2023

(51) Int. Cl. A61M 37/00; A61K 9/16; A61K 31/337; A61K 31/436; A61K 31/704; A61M 5/315; A61M 5/42

(52) U.S. Cl. CPC A61M 37/0069; A61K 9/1647; A61K 31/337; A61K 31/436; A61K 31/704; A61M 5/3157; A61M 5/31585; A61M 5/427; A61M 2205/581; A61M 2205/582; A61M 2210/166

(57) ABSTRACT

Minimally invasive treatment methods for benign prostatic hyperplasia (BPH) tissue are disclosed. A system includes a sustained release solid formulation comprising a cytostatic or cytotoxic drug, and an applicator or delivery system for local delivery of a composition comprising the sustained release solid formulation to the prostate.

### TECHNICAL FIELD

Minimally invasive, local treatments for men's health and, more particularly, lower urinary tract symptoms, including benign prostatic hyperplasia.

### BACKGROUND

Enlarged prostate or benign prostatic hyperplasia (BPH) negatively affects the quality of life in 60% of men over the age of 60 and 90% of men over the age of 70. BPH currently impacts more than 14 million men in the United States and about 94 million men globally. In the U.S. alone, BPH represents a $20 billion market opportunity. The symptoms of this growing healthcare challenge include urinary storage and voiding issues including retention. When left untreated BPH can lead to sexual dysfunction, bladder and kidney damage, and ultimately death. Beyond the significant negative impact on quality of life, BPH is one of the costliest and most frequent treatment segments of urology. Current treatments, such as oral drugs, however, have limited efficacy, require long term compliance, and cause significant side effects. Surgery and other procedures are invasive and also have risks of significant side effects.

### SUMMARY

In some aspects, the presently disclosed subject matter provides a preformed injectable solid implant for treating benign prostatic hyperplasia (BPH), the implant comprising one or more therapeutic agents in a bioabsorbable polymer, wherein the implant has a shape selected from a rod, spheroid, grain, and pellet, and a size in a first dimension (a) between about 0.3 mm and about 6 mm and a size in a second dimension (b) between about 0.2 mm and about 1.5 mm, provided that the size of the first dimension (a) is greater than the size in the second dimension (b). In certain aspects, the bioabsorbable polymer comprises poly(lactide-co-glycolide) (PLGA) or poly(D,L-lactide) (PDLA). In certain aspects, the one or more therapeutic agents comprise paclitaxel or sirolimus.

In other aspects, the presently disclosed subject matter provides a method for treating BPH in a subject in need of treatment thereof, the method comprising administering a solid implant as disclosed hereinabove at a first prostate location of the subject. In certain aspects, the implant is administered by injection through an abdomen, rectum, a perineum, or an urethra of the subject. In certain aspects, the first prostate location comprises a transition zone of the subject's prostate. In certain aspects, the implant is administered via a needle, in some aspects, guided by ultrasound imaging. In certain aspects of the method, a plurality, e.g., up to about 20 implants, can be administered to the prostate. In certain aspects, the implant is administered by injection or implantation through a rectum, a perineum, or a urethra of the subject.

In yet other aspects, the presently disclosed subject matter provides a system for treating BPH in a subject in need of treatment thereof, the system comprising: (i) one or more implants as described hereinabove; and (ii) an applicator or delivery system for local delivery of the one or more implants to target tissue of the subject's prostate. In certain aspects, the applicator or delivery system comprises a needle, wherein the needle has one or more characteristics selected from: (i) a length from about 15 cm to about 20 cm; (ii) a lumen having a diameter from about 0.02 cm to about 0.12 cm; and (iii) a ratio of length divided by lumen diameter of between about 200 and 400, between about 100 and 1000, or between about 125 and 1000. In particular aspects, the system further includes an ultrasound device for locating target tissue in the prostate, or an echogenic device more visible by ultrasound in the target prostate tissue.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

Having thus described the presently disclosed subject matter in general terms, reference will now be made to the accompanying drawings, which are not necessarily drawn to scale, and wherein:

FIG. 1 is a representative embodiment illustrating injection of a plurality of the presently disclosed implants 5 into the transition zone 110 of the prostate 100 of a subject using an injection device, e.g. injection device 20;

FIG. 2A is a photograph of an exemplary rod-shaped preformed solid implant 5;

FIG. 2B is an exemplary rod-shaped preformed solid implant 5a having a first dimension a and a second dimension b;

FIG. 2C is an exemplary capsule-shaped preformed solid implant 5b having a first dimension a and a second dimension b;

FIG. 2D are representative shapes of the presently disclosed implants 5, including an oblate spheroid, a prolate spheroid, and grain shapes, including well rounded, rounded, subrounded, subangular, angular, and very angular grain shapes;

FIG. 3 shows a representative delivery device 300 for delivering the presently disclosed implants 5 to prostate 100 via a transperineal, transrectal or transurethral route using a plurality of implant catheters 310 affixed to template 320 under transrectal ultrasound imaging with an ultrasound probe 330;

FIG. 4 shows a first needle 12 and plunger portion 14 of a medical device 10 for injection of the presently disclosed implants 5;

FIG. 5 shows a second needle 12 and plunger portion 24 of a medical device 20 for injection of a plurality of the presently disclosed implants 6; and

FIG. 6A shows a handle and control 30 of a medical device according to one example;

FIG. 6B shows a handle and control portion 40 of a medical device according to one example; and

FIG. 6C shows a handle and control portion 50 of a medical device according to one example.

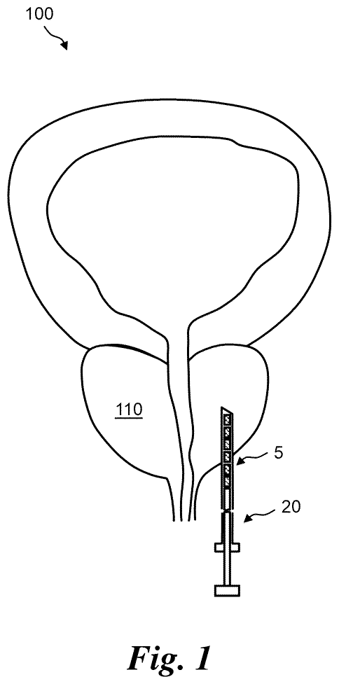

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Development of a cost-effective, drug-eluting therapy that reduces pressure, through tissue atrophy, on the compressed urethra of a patent afflicted with BPH could transform treatment of BPH by dramatically increasing quality of life for a significant growing population worldwide. Accordingly, in some embodiments, the presently disclosed subject matter provides an intraprostatic solid drug-eluting bioabsorbable implant therapy, aimed for the prostate transition zone, which creates tissue atrophy through sustained elution, for example, of antiproliferative and/or cytotoxic drugs including, but not limited to, paclitaxel, docetaxel, cabazitaxel, doxorubicin, and the like. Alternatively, cytostatic, such as sirolimus, everolimus, zotarolimus, and the like, anti-inflammatory, anti-fibrotic, or anti-androgenic drugs can be used with the presently disclosed implants. Generally, any drug or therapeutic agent that would affect the prostate size and gland proliferation is suitable for use with the presently disclosed implants and methods. In common for all these therapeutic agents is that local delivery will reduce overall dose and systemic exposure, thereby significantly reducing known side effects.

Sustained release of the drug over time also will reduce the need for patient compliance to achieve the desired outcomes. Further, a sustained release of the drug over time will keep the tissue concentrations of the drugs consistent for improved efficacy and reduced side effects. In some embodiments, the drug can be released from the solid implant in a slow, sustained release fashion, optionally delivered as an initial burst of the drug, followed by a slow, sustained release of the drug to the target release. The amount or lack of burst and/or the “slow, sustained release” release period may depend on the drug delivered to the prostate, the solid implant properties, as will be appreciated in view of this disclosure. In some embodiments a slow, sustained release may occur over, e.g., a 24-hour period, 3-7 days, 1-4 weeks, 1 to 12 months, approximately 3 months, approximately 6 months, approximately 6 to 12 months or approximately 12-24 months.

To this end, the presently disclosed subject matter includes drug-eluting implants, the delivery system, and clinical methods of their application. Particular embodiments of the presently disclosed implants include the size, shape and number of implants, as well as the drug dose and target sustained release rate, which can be controlled by the composition of the drugs and polymers used, drug-to-polymer ratios, polymer molecular weight, polymer end groups, polymer degradation rate, and/or the polymer and/or drug morphology.

Injectable and bioabsorbable sustained release local drug delivery implants for BPH that are generated in-situ are disclosed in U.S. Pat. No. 11,602,516 and U.S. Patent Application Publication No. 2022/0031606A1, respectively, each of which is incorporated herein by reference in its entirety. The formulation disclosed therein creates prostate tissue atrophy by eluting, for example, paclitaxel, over a period of between about one to three months. The implant comprising bioabsorbable polylactide-co-glycolide (PLGA) 50/50 degrades into water and carbon dioxide over a similar time period.

The formulation disclosed in U.S. Pat. No. 11,602,516 and U.S. Patent Application Publication No. 2022/0031606A1 comprises paclitaxel dissolved in a viscous gel, which is created by dissolving PLGA 50/50 in N-methyl pyrrolidone (NMP). The in-situ generated implants are created instantly when the formulation comes into contact with interstitial fluids in the prostate, forcing the drug and the polymer to precipitate out of the formulation as the water soluble NMP is mixed with the interstitial fluids. Each injectate, which is between approximately 50 μL to 250 μL per unit volume, creates individual micro small implants designed to release the drug with close to first order sustained release over the desired period of time, for example 50% released in the first month and the remainder released within three months of implantation.

In contrast to the viscous, liquid formulation disclosed in U.S. Pat. No. 11,602,516 and U.S. Patent Application Publication No. 2022/0031606A1, the presently disclosed subject matter provides an injectable and/or implantable solid implant. The solid implant is designed to be delivered to the transition zone of the prostate through long narrow/slender needles (e.g., 20-cm long with a 0.060-0.085-cm lumen size), which facilitate injection through the rectum, perineum, or urethra using ultrasound guidance without creating the unnecessary risk of infection that is typical for large bore needles and to better control and retain the amount of solid implant material injected at one injection site. Further, the proper dose and drug release rate can be controlled to create drug tissue lesions large enough to eliminate or at least reduce the pressure on the urethra and, at the same time, keep the drug in the prostate without adversely impacting or damaging surrounding organs, such as the bladder, urethra, testicles, and the seminal vesicles. In some embodiments the needle lumen diameter is between 2-5% higher than the average diameter of the implant.

To obtain an atrophy volume sufficient to reduce urethral pressure, multiple small implants are deposited in each lobe of the prostate. Each implant creates a surrounding drug lesion. The size of each of these drug lesions is controlled by the overall drug flux from the implants. The drug flux is controlled by multiple factors, including the drug dose, the molecular structure of the drug (e.g., molecular weight, lipophilicity, crystallinity) and polymer (e.g., molecular weight, monomer ratios, degradation rate, and the like), the concentration of both the polymer and drug used, and the drug/polymer ratio.

Representative components of formulations suitable for use with the presently disclosed subject matter are disclosed, in part, in U.S. Pat. No. 11,602,516, col. 9, line 15 through col. 11, line 20, which is incorporated herein by reference in its entirety. Note that the formulations in U.S. Pat. No. 11,602,516, however, are a viscous liquid injectate, which is distinguishable from the currently disclosed preformed injectable solid implants.

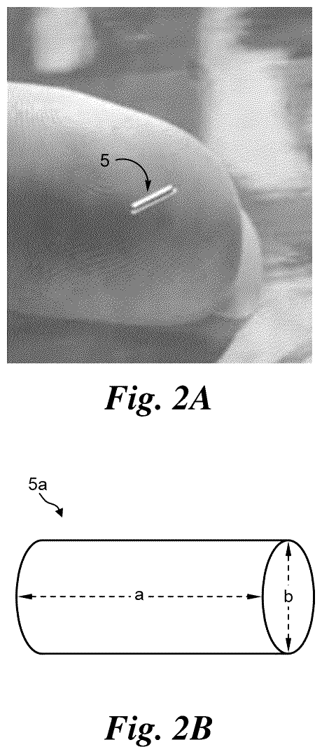

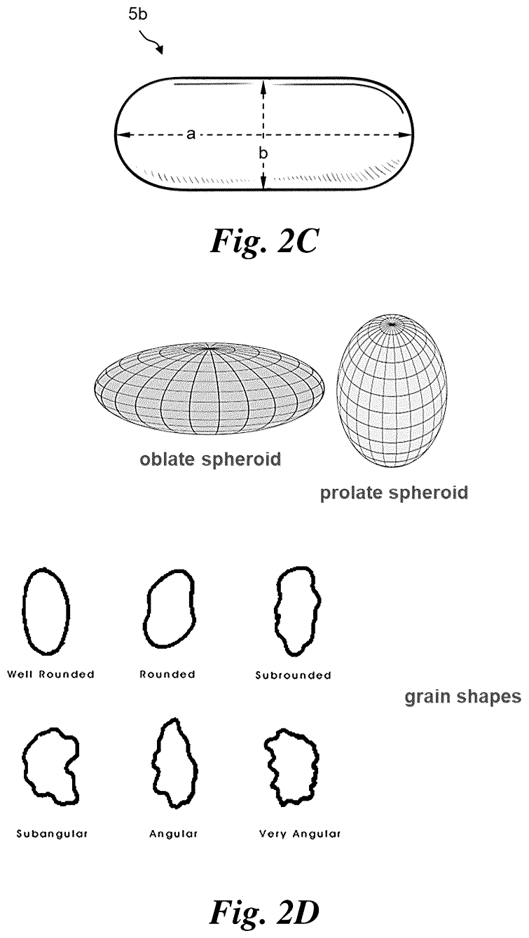

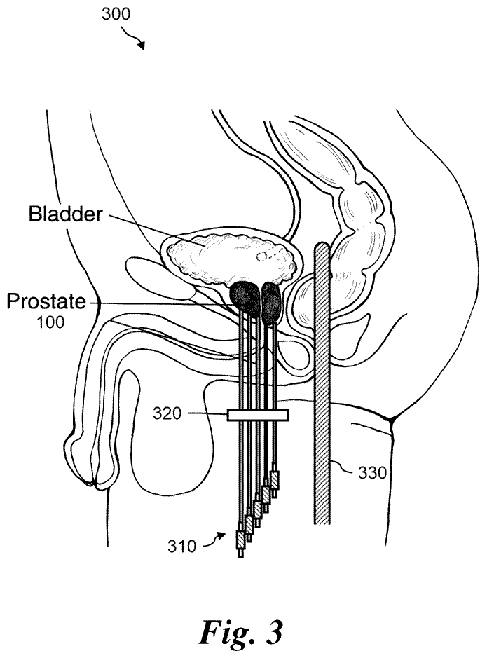

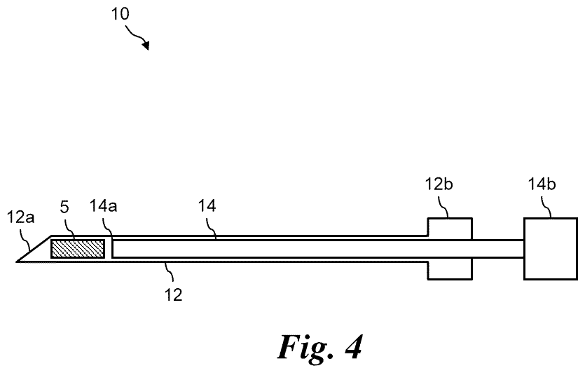

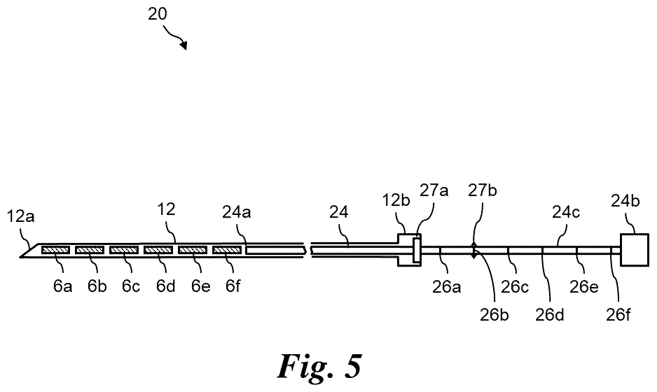

FIG. 3 shows one example of a medical device 300 for delivering a plurality of the implants 5 (FIGS. 2A-2C) to a plurality of targeted sites in the prostate 100 (FIG. 3 shows the needles accessing the prostate via a transperineal route). It is contemplated that multiple implants, e.g., 2 up to 20 implants, can be delivered to the prostate transition zone in a patient with BPH using medical device 300. Referring once again to FIG. 3, medical device 300 includes a plurality of implant needles 310 operationally affixed to template 320. The individual needles may be the needle portion of the medical device 10 or 20 (FIGS. 4 and 5, respectively). Some embodiments of the use of medical device 300 include guidance using transrectal ultrasound imaging, for example, with an ultrasound probe 330.

Alternatively, a single needle may be placed in multiple positions in the prostate for delivery of implants at different targeted sites. FIG. 4 and FIG. 5 are partial views of two such medical devices.

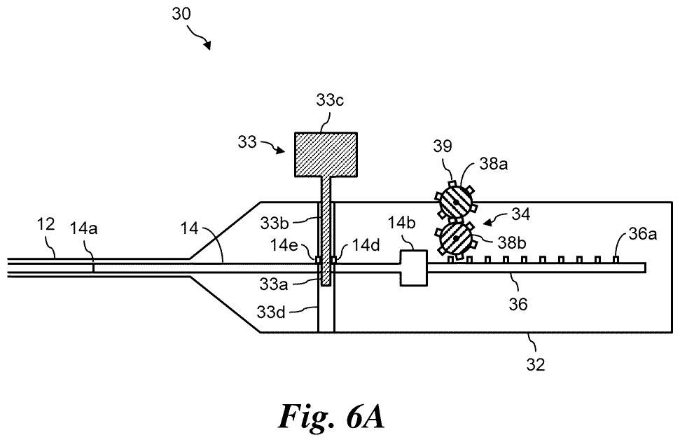

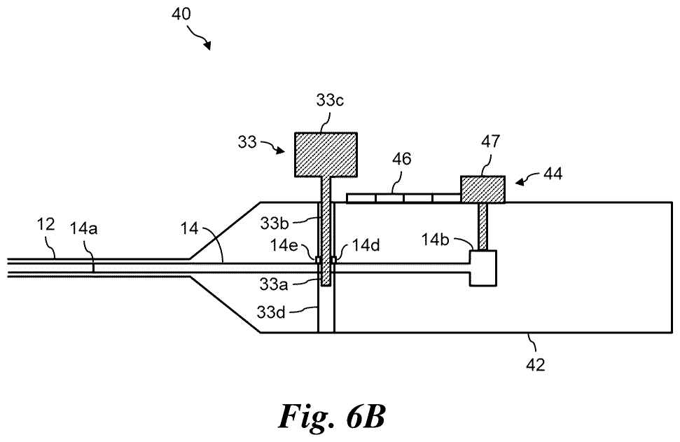

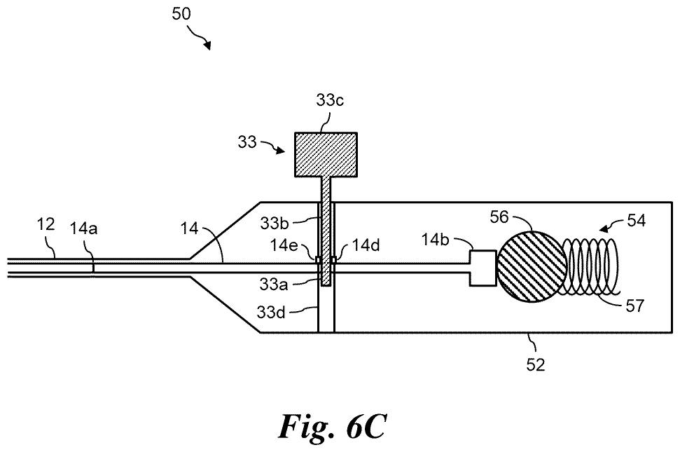

FIG. 4 shows a first needle and plunger portion of a medical device 10, which is configured to deliver a single implant per injection site. Several of the medical devices 10 may be coupled to the template to make the medical device 310 shown in FIG. 3. Needle 12 has a proximal end forming a hub 12b and a distal end 12a forming a needle bevel. A single implant 5 is pre-loaded in the needle's lumen before the needle is placed at the distal implant site. The implant 5 is located between the needle tip 12a and a distal end 14a of a plunger 14. A neck of the plunger 14 is disposed within the lumen of the needle 12 and movable to and from the distal end 12a. At the proximal end of the needle 12 and plunger 14 is a respective a needle hub 12b and a plunger hub 14b, examples of which are shown in FIGS. 6A, 6B and 6C. The plunger 14 neck has a diameter slightly smaller than the needle bore diameter, so that it may move smoothly within the bore and yet easily direct the implant 5 toward the tip 12a as the needle is withdrawn from the distal implant site.

FIG. 5 shows a second needle and plunger portion of a medical device 20, which is configured to deposit a plurality of implants 6 in the needle track as the needle is withdrawn from the distal implant site, and/or to deposit a plurality of implants at a plurality of implant sites. The needle and plunger are the same as described above where the same element numbering is used in FIG. 5. For the portion of a medical device depicted in FIG. 5, there are a plurality of pre-loaded implants (e.g., implants 6a, 6b, 6c, 6d, 6e, and 6f) located adjacent the distal end 12a and between the tip 12a and plunger tip 24a. A proximal neck 24c of the plunger 24 has indicia (e.g., indentions, markers and/or detents) used to guide the release or dispensing of each implant from the needle distal end as the needle 12 is withdrawn from a distal implant site. For example, needle 12 is pre-loaded with the six implants 6a, 6b, 6c, 6d, 6e, and 6f as shown. There are six corresponding indicia (e. g. 26a, 26b, 26c, 26d, 26e, 26f) found on the plunger neck portion 24c extending between the needle hub 12b and plunger hub 14b. As each implant is released from the needle, the indicia displace to the left, indicating the release of the corresponding implant at the distal end 12a. A health professional may use the indicia as a guide to verify the release of one or more of the implants at the injection site. The indicia may be a marker to visually indicate the release of an implant. The indicia may also, or alternatively, provide an audible or other sensory output (finger pressure) when an implant is released. This may be done by forming a pair of detents 27b, 27a with the detent 27a resisting movement of the plunger from right to left until the needle-hub detent 27a is overcome by finger pressure (i.e., finger pressure that tends to pull the needle hub toward the plunger hub (audible “clicking” sound and/or sudden loss in resistance to the pulling motion when the pair of detents have cleared each other). The plunger neck-detent 27b would be formed on each of the indicia locations (i.e., each of 26a, 26b, 26c, 26d, 26e, and 26f include a detent) in the illustrated example.

According to a method using a medical device having the needle and plunger of FIG. 4 or FIG. 5, the needle tip is first located at the distal implant site, or site targeted by the health professional for injection of one or more implants. Once there, the needle hub is pulled toward the plunger hub by finger pressure or an alternative created pressure, thereby withdrawing the needle tip 12a from the distal implant site, which releases the implant 5 or 6a in the needle track. Referring to FIG. 6, the health professional may, after dispensing implant 6a, continue to pull the needle hub 12b toward the plunger hub 24b to release additional implants at the targeted site. The total number of implants dispensed at a single target site in the prostate depends on the total volume for treatment (i.e., the number of implanted implants corresponding to a volume of implanted composition producing an efficacious outcome, that is, significant reduction in prostate volume) or the anatomy and/or shape of the obstruction at that target site in the prostate. It is contemplated that the multiple implant injection device of FIG. 5 may be constructed to carry and dispense accurately (one at a time) 6 or more (e.g., 6, 7, 8, 12, 15) implants at single implant site. Desirably, the size of the implants (millimeter size, as opposed to micro or nano-sized particles) enables an accurate metering of the implants to achieve the localized tissue atrophy sites needed for individual target sites in the prostate. Micro- or Nano-sized particles, by contrast, have dimensions that are too small for metering using a needle as a solid implant. Rather, the drug load delivered for these types of implants can only be metered by estimating the number of particles per volume of liquid in which the particles are suspended and contained within a syringe barrel. Moreover delivery of micro- or nano-sized particles is considered uncontrolled and may not remain or target only in the prostate in contrast to delivery of a millimeter sized solid implant by this invention to remain in the prostate.

FIGS. 6A, FIG. 6B, and FIG. 6C show embodiments of a handle and control portion of a needle injection device for injection of the presently disclosed implants at one or more target sites of the prostate. The distal ends of medical devices (i.e., needle distal end 12a and plunger end 14a) configured with either handle and control 30 or handle or control 40 will have a plurality of implants pre-loaded at the distal end 12a, similar to what is shown in FIG. 5, but with proximal ends operated differently than described with reference to FIG. 5.

FIG. 6A shows a handle and control portion 30 of a medical device according to one example, including a safety lock 33 and a rotary mechanism 34 at least partially contained within a housing 32. The needle 12 proximal end is secured at a distal end of the housing 32 of the handle and control 30. The plunger 14 is received in the needle bore and extends into the housing 32. The rotary mechanism 34 operated by finger pressure advances the plunger toward the needle distal end as a user grips the handle housing 32.

The rotary mechanism 34 includes a longitudinal piece 36 (which may simply be an extension of the hub 14), and wheels 38a, 38b that can be turned by a finger pressure applied to knobs 39 that are external to the housing (for accessing by the user). Piece 36 extends toward the proximal end of housing 32. Piece 36 includes knobs 36a engaged with mating gear teeth on a rotary wheel 38b mounted to the housing 32. The rotary wheel 38b is in-turn engaged with a second wheel 38a (half of which extends outside the housing 32) that has formed thereon mating gear teeth 39 that can be gripped by finger pressure such that a user can rotate the wheel 38b using a finger or thumb (i.e., a thumb wheel). As the wheel is rotated counterclockwise by the user, the 38b rotates clockwise, which in turn causes the gear teeth to engage knobs 36a to push piece 36 and therefore plunger distal end 14a toward the needle distal 12a to release an implant. Each turn of the wheel through a designated angle may be registered to the plunger distance end 14a advancing by a distance needed to dispense one implant from the needle distal end 12a. The movement through this angle may be indicated by an audible sound (a “click” caused by a detent mechanism between the housing 32 and wheel 38a). Thus, with each clocking of the wheel 38a through the predetermined angle (indicated by an audible click and/or resistance to turning, followed by a free rotary movement) the user dispenses one implant from distal end 12a while gripping the housing 32 and applying finger pressure or an alternative pressure to knob 39. Thus, for a needle preloaded with 6 implants, there are a corresponding (at least) 6 available clocking of the wheel, each clocking corresponding to the forward movement that will dispense an implant from the needle 12a distal end.

Handle and control portion 30 includes a safety lock 33 to prevent unintended movement of the plunger 14. Safety lock 33 includes a knob 33c and arm 33b having an opening 33a through which the plunger 14 extends. In the locked position (as shown) the plunger 14 may not move to the left or right due to the presence of knobs 14e, 14d on opposite sides of arm 33b. When the knob 33c of the safety lock 33 is pulled upward (as indicated by the arrow), the opening 33a is moved upward to a position clearing arm 33b from the knobs 14d, 14e. Once clear, the plunger 14 may be moved forwardly (toward the distal end 12a of the needle 12) by finger pressure applied to the knobs of wheel 38a. The wheel 38a may be clocked through a consecutive number of angles corresponding to the number of implants pre-loaded at the needle distal end 12a.

FIG. 6B shows a handle and control portion 40 of a medical device according to one example, including a safety lock 33 and a rotary mechanism 44 at least partially contained within a housing 42 (e.g., a rack and pinion device could be used to control the rotary mechanism). The medical device depicted in FIG. 6B is similar to that shown in FIG. 6A. It also has the safety lock 33 operated in the same manner as the handle and control portion 30. Rather than a rotary mechanism 34, connected however, handle and control portion 40 uses a slider mechanism 44 to advance the plunger toward the needle distal end 12a. The slider mechanism includes a longitudinal track 46 receiving a post 47a, slidable along the track. The post 47a has at one end a finger actuated button 47 and, at the opposite end (within the housing 42) the post 47a is to the plunger hub 14b. Movement of the button 47 forward by finger pressure correspondingly moves the plunger 14 forward. The track 46 includes a plurality of indicia 46a registered to the forward movement that will dispense an implant. Thus, for a needle preloaded with 6 implants, there are a corresponding (at least) 6 indicia located on the track, each indicia separated by a distance corresponding to the forward movement that will dispense an implant from the needle 12a distal end. The indicia may be one or more of a visual, audible (click) or finger-pressure sensed to indicate movement causing an implant to dispense from the needle.

FIG. 6C shows a handle and control portion 50 of a medical device according to one example. The medical device depicted in FIG. 6C is similar to that shown in FIG. 6A and FIG. 6C. It also has the safety lock 33 operated in the same manner as the handle and control portion 30 and 40. Rather than a rotary mechanism 34 or slider mechanism 44 to advance the plunger toward the needle distal end 12a, the device depicted in FIG. 6C is spring activated and includes housing 52, comprising spring loaded button 56, which can be used to activate spring 57 in a longitudinal direction 54 to deliver the solid implant.

The embodiments described with reference to FIG. 5 and FIG. 6A-6C are examples of a medical device portion of an applicator or delivery system. This system includes a plurality of implants 5. In the illustrated examples implants 5 are shown contained within a lumen of the needle 12. By pressure applied to an implant 5 through a plunger 14 individual implants may be individually ejected from a tip of the needle 12 (not shown). It is desirable, for the purposes of ejecting individual implants from the needle tip one at a time in a controlled manner and without damaging the implant, to avoid causing an implant to rotate/displace off-axis when pressure is applied by the plunger (i.e., rotate up or down within the needle lumen to cause it to catch or fracture by eccentric pressure applied to the implant 5). That is, within the lumen when pressure is applied to an implant 5, one wants to have the implant displace only along the lumen axis of the needle. To better ensure this happens when pressure is applied to an implant 5, the lumen has a size between 2% and 10%, 5% and 15%, 5% and 10%, or 6% to 8% greater than a dimension of the implant (e.g., a diameter, in the case of a cylindrical implant shape). In some embodiments the surface of the lumen may be coated to lower the surface friction between implant and lumen when pressure is applied to implant 5.

In alternative embodiments, the applicator or delivery system may include a cartridge containing a plurality of the implants 5. The cartridge would be attached to the control and handle portion with a lever of the cartridge actuable to release one or more of a plurality of implants into the needle lumen forward of the plunger. Alternatively, the handle or control portion may include a chamber for receiving from the cartridge individual ones of the implants, which are then individually placed within the needle lumen 12, or another lumen associated with the applicator or delivery system (selectively operated to dispense implants into the lumen of the needle 12).

The presently disclosed subject matter provides a BPH therapy using similar drugs as used in U.S. Pat. No. 11,602,516 and U.S. Patent Application Publication No. 2022/0031606A1. In contrast to the therapies disclosed in U.S. Pat. No. 11,602,516 and U.S. Patent Application Publication No. 2022/0031606A1, the instant disclosure is directed to solid implants, which are preformed and delivered to the prostate transition zone as small rods, grains, spheroids or pellets, as shown in FIG. 2. It is important to note that the presently disclosed solid implants are millimeter sized (greater than or equal to 1×10⁻³ m in dimension) and do not include microparticles, e.g., particles having a dimension of about 0.1 μm to about 100 μm, and less than 1000 μm, or nanoparticles, e.g., particles having a dimension of about 1 nm and 100 nanometers, and less than 1000 nm, and have a range of dimensions larger than micro- or nanoparticles as provided herein below. Micro- or nanoparticles are subject to the limitation that their delivery to the prostate is not controlled and they may migrate to surrounding tissues and organs such as bladder, testes, seminal vesicles and urethra causing unwanted damage and resulting in sub-therapeutic drug concentrations in the prostate.

In contrast to micro-particles or nanoparticles, in some embodiments, the solid implants are not delivered using a liquid medium. The solid implants are not suspended in a liquid medium. Instead, the solid implants are released from the needle by a contact force (spring, stylet) that pushes the implant toward the needle distal end while the needle is located at and/or being retracted from the target site. In other embodiments, the implants are pre-loaded in a needle tip, then a syringe with a liquid is connected to the pre-loaded needle. The liquid is used to apply pressure to the implants using the syringe plunger to release each implant, one at a time. In this embodiment as well, the implant is not suspended in the liquid, Rather, the liquid is used to apply pressure to release each implant from the distal end. Another embodiment would have the implants preloaded in a second lumen and would be charged individually into the needle lumen with the syringe plunger or stylet used to release each implant. This would help to better control the exact location of each implant delivered.

The dimensions of the implants ideally should be small enough for delivery in a metered fashion, that is, one at a time, through a small-bore needle to reduce the risk of infection if delivered transrectally and to reduce trauma if delivered in an alternative fashion. The implants should be large enough for handling during manufacturing and to allow therapeutic sustained release of sufficient dosage of hydrophobic drugs, such as paclitaxel or sirolimus.

The drug release rate of polymeric implants can be first order, close to zero order, and or multiphasic and is typically controlled by either polymer degradation, drug diffusion, water uptake, polymer glass transition temperature, polymer topcoat (for zero order release) or a combination thereof. The polymer molecular structure also is important as it controls implant morphology, drug miscibility, water uptake, and, for bioabsorbable polymers, also the degradation rate. The implant morphology also is controlled by process techniques. In some embodiments, the presently disclosed implants are prepared using solvent casting or solvent extrusion to minimize degradation of small molecule drugs, as well as minimize resulting drug crystalline morphology for appropriate first order drug release profiles.

The polymers used for the implants with sustained drug release controlled by degradation would ideally be amorphous and, in some embodiments, PLGA-based using enough lactide or glycolide for rapid degradation. PLGA (poly(D,L-lactide-b-glycolide) polymers with a lactide: glycolide ratio of about 50:50, 65:35, 75:25, 85:15, and 10:90 are suitable for use with the presently disclosed implants. The PLGA also can be a block copolymer with poly(ethylene glycol) (PEG) to allow for faster water uptake and more rapid degradation. These materials would allow for complete bio absorption of the implants leaving no permanent foreign body left behind in comparison to permanent polymers, such as silicones, polyurethanes, and the like.

Alternatively, semicrystalline or amorphous bioabsorbable polymers can be designed to have low glass transition temperatures (Tg) of the amorphous regions specifically designed to dissolve drugs, such as paclitaxel or sirolimus. Using these block copolymers, the drugs can diffuse through the polymer even at low drug to polymer ratios. The Tg of these polymers also can be designed to be reduced as they absorb water allowing the drugs to diffuse out.

Properties of representative implants are provided in Table 1 and Table 2.

TABLE 1
Properties of Representative Solid Cylindrical Implants

| | | | | Drug (mg)/ | |
|---|---|---|---|---|---|
| Implant Dimensions | | | | Implant (mg) | |
| Length | Diameter | Volume | Weight | Drug/Polymer | Drug (mg)/ |
| (mm) | (mm) | (mm³) | (mg) | (1/9) | 20 Implants |
| 6 | 1 | 4.71 | 5.89 | 0.59 | 11.8 |
| 5 | 1 | 3.93 | 4.91 | 0.49 | 9.8 |
| 4 | 1 | 3.14 | 3.93 | 0.39 | 7.8 |
| 3 | 1 | 2.36 | 2.95 | 0.30 | 6.0 |
| 2 | 1 | 1.57 | 1.96 | 0.20 | 4.0 |
| 6 | 0.8 | 3.02 | 3.77 | 0.38 | 7.6 |
| 5 | 0.8 | 2.51 | 3.14 | 0.31 | 6.2 |
| 4 | 0.8 | 2.01 | 2.51 | 0.25 | 5.0 |
| 3 | 0.8 | 1.51 | 1.88 | 0.19 | 3.8 |
| 2 | 0.8 | 1.01 | 1.26 | 0.13 | 2.6 |
| 6 | 0.6 | 1.70 | 2.12 | 0.21 | 4.2 |
| 4 | 0.6 | 1.13 | 1.41 | 0.14 | 2.8 |
| 3 | 0.6 | 0.85 | 1.06 | 0.11 | 2.1 |

For implants loaded with one or more therapeutic agents, e.g., paclitaxel or sirolimus, with a size (weight) of between approximately 1 mg to 6 mg and a drug to polymer ratio of 1 to 10 to 6 to 10, and, depending on the size of the implants, a total drug load up to approximately 2 to 12 mg paclitaxel/patient when using a total of 20 implants at a drug to polymer ratio of 1 to 10 is anticipated. See Table 1. The created lesions are estimated to have a dimension of approximately 10 mm×20 mm or more and that up to 10 implants can be delivered in each prostate lobe depending on the size of the enlarged prostate. This estimate assumes a density of 1.25 g/mL for the implant.

For implants loaded with one or more therapeutic agents, e.g., paclitaxel or sirolimus, with a size (weight) of between approximately 1 mg to 6 mg and a drug to polymer ratio of 3 to 5 and depending on the size of the implants, it is expected that a total drug load up to approximately 71 mg paclitaxel/patient when using a total of 20 implants can be achieved. See Table 2. The created lesions are estimated to be approximately 10 mm×20 mm and that up to 10 implants could be delivered in each prostate lobe pending the size of the enlarged prostate. This estimate assumes a density of 1.25 g/mL for the implant.

TABLE 2
Properties of Representative Implants

| | | | | Drug (mg)/ | |
|---|---|---|---|---|---|
| Implant Dimensions | | | | Implant (mg) | |
| Height | Diameter | Volume | Weight | Drug/Polymer | Drug (mg)/ |
| (mm) | (mm) | (mm³) | (mg) | (3/2) | 20 Implants |
| 6 | 1 | 4.71 | 5.89 | 3.53 | 70.7 |
| 5 | 1 | 3.93 | 4.91 | 2.95 | 58.9 |
| 4 | 1 | 3.14 | 3.93 | 2.36 | 47.2 |
| 3 | 1 | 2.36 | 2.95 | 1.77 | 35.4 |
| 2 | 1 | 1.57 | 1.96 | 1.18 | 23.5 |
| 6 | 0.8 | 3.02 | 3.77 | 2.26 | 45.2 |
| 5 | 0.8 | 2.51 | 3.14 | 1.88 | 37.7 |
| 4 | 0.8 | 2.01 | 2.51 | 1.51 | 30.1 |
| 3 | 0.8 | 1.51 | 1.88 | 1.13 | 22.6 |
| 2 | 0.8 | 1.01 | 1.26 | 0.76 | 15.1 |
| 6 | 0.6 | 1.70 | 2.12 | 1.27 | 25.4 |
| 4 | 0.6 | 1.13 | 1.41 | 0.85 | 16.9 |
| 3 | 0.6 | 0.85 | 1.06 | 0.64 | 12.7 |

More particularly, in some embodiments, the presently disclosed subject matter provides a preformed injectable solid implant for treating benign prostatic hyperplasia (BPH), the implant comprising one or more therapeutic agents in a bioabsorbable polymer, wherein the implant has a shape selected from a rod, a spheroid, including an oblate spheroid or a prolate spheroid, a grain, and a pellet, and a size in a first dimension (a) between about 0.3 mm and about 6 mm and a size in a second dimension (b) between about 0.2 mm and about 1.5 mm, provided that the size of the first dimension (a) is greater than the size in the second dimension (b), that is a>b.

In certain embodiments, the size of the first dimension (a) and the size of the second dimension (b) is selected from about 6 mm×1.5 mm, 4 mm×1.5 mm, 3 mm×1.5 mm, 2 mm×1.5 mm, 6 mm×1 mm, 4 mm×1 mm, 3 mm×1 mm, 2 mm×1 mm, 1.5 mm×1 mm, 6 mm×0.5 mm, 4 mm×0.5 mm, 3 mm×0.5 mm, 2 mm×0.5 mm, 1.5 mm×0.5 mm, 1 mm×0.5 mm, 6 mm×0.4 mm, 4 mm×0.4 mm, 3 mm×0.4 mm, 2 mm×0.4 mm, 1.5 mm×0.4 mm, 1 mm×0.4 mm, 6 mm×0.3 mm, 4 mm×0.3 mm, 3 mm×0.3 mm, 2 mm×0.3 mm, 1.5 mm×0.3 mm, 1 mm×0.3 mm, 0.5 mm×0.3 mm, 6 mm×0.2 mm, 4 mm×0.2 mm, 3 mm×0.2 mm, 2 mm×0.2 mm, 1.5 mm×0.2 mm, 1 mm×0.2 mm, 0.5 mm×0.2 mm, and 0.3 mm×0.2 mm.

In certain embodiments, the implant has a weight having a range from about 1 mg to about 6 mg, including about 1.0 mg, 1.5 mg, 2.0 mg, 2.5 mg, 3.0 mg, 3.5 mg, 4.0 mg, 4.5 mg, 5.0 mg, 5.5 mg, and 6.0 mg.

Factors and characteristics influencing the development of the presently disclosed solid implant include, but are not limited to:

Drug to Polymer Ratio. Too low of a drug to polymer ratio is not optimum. If the drug dosage is too low, then it results in the local drug concentrations being too low. Too high of a drug to polymer ratio results in poor mechanical integrity of the implant for manufacturing and may result in the drug releasing from the solid implant too fast, which limits the longer term local drug release and also leads to surrounding tissue and/or systemic drug exposure. It is desired to have a controlled, sustained release of, e.g., from 14 days or 1 to 24 months for substantially all of the drug to dissipate from the solid implant. This characteristic requires the selection of the polymer composition and/or modifying the morphology and mechanical properties, the polymer/drug ratio, controlling the physical shape/dimensions (volume) of the solid implant and/or composition delivered to the target tissue.

Other factors affecting the release rate include, but are not limited to, ability of the polymer to swell (controlled by polymer structure, e.g., monomers selection, monomer ratios, molecular weight, end groups, and porosity/morphology); porosity/morphology (controlled by, e.g., polymer structure and concentration, and polymer/drug ratio-less drug than polymer than drug is trapped); polymer degradation (controlled by polymer structure e.g., monomers selection, monomer ratios, molecular weight, end groups, and porosity/morphology); drug/polymer miscibility; and molecular weight and lipophilicity of drug.

Drug or drug combinations used in the solid implant include a cytostatic drug, cytotoxic drug, and/or other drugs. The other drug(s) may be used by themselves (i.e., the “other drug(s)” are the only active agents in the solid implant), or in combination with the cytostatic drug or cytotoxic drug as part of the medical procedure for treatment of BPH. For example, the other drug(s) may be administered before the solid implant is injected into the target tissue using a needle syringe, or the other drug(s) may be included in the solid implant. In other embodiments, the other drug(s) may be administered after the solid implant containing the cytostatic drug or cytotoxic drug is administered to the target tissue.

Representative other drugs that may be administered with, or instead of the cytostatic or cytotoxic drug, include, but are not limited to, alpha blockers, 5-alpha reductase inhibitors, or nonsteroidal antiandrogens. Alpha blockers may include and are not limited to terazosin, doxazosin, tamsulosin, alfuzosin, and silodosin. 5-alpha reductase inhibitors may include and are not limited to finasteride and dutasteride. Anti-inflammatory drugs may include but are not limited to corticosteroids, such as dexamethasone, fluticasone propionate, triamcinolone acetonide, mometasone furoate, prednisone, hydrocortisone, estradiol, clobetasol, and budesonide. Non-steroidal drugs may include acetaminophen, ibuprofen, and naproxen. These other drug types may block cytokine activity or inhibit binding of cytokines to inhibit inflammatory signals such as anti-IL1, anti-IL 2, anti-IL3, anti-IL4, anti-IL8, anti-IL15, anti-IL 18, anti-MCP 1, anti-CCR2, anti-GM-CSF, anti-TNF antibodies and others. Nonsteroidal antiandrogens may include and are not limited to aminoglutethimide, apalutamide, bicalutamide, enzalutamide, flutamide, ketoconazole, nilutamide, and topilutamide.

Five alpha reductase inhibitors reduce the prostate volume by 20% when given orally. Minimal reduction occurs in less than six months. An up to 20% reduction in prostate volume is expected in 6-24 months or possibly longer with appropriate therapy.

Alpha blockers can also be used to treat symptomatically at the time of procedure by blocking the alpha receptor and relaxing the prostate smooth muscle. Alpha blockers, five alpha reductase inhibitors, nonsteroidal antiandrogens or all may be co-formulated with cytostatic or cytotoxic drugs in the solid implant.

In certain embodiments, the one or more therapeutic agents have a dose ranging from about 0.1 mg to about 2 mg, including about 0.1 mg, 0.2, mg, 0.3, mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8, mg, 1.9 mg, and 2.0 mg.

In certain embodiments, the implant comprises a plurality of implants having a total dose of one or more therapeutic agents ranging from about 2 mg to 70 mg or about 10 mg to 20 mg, including about 10 mg, 11 mg, 12 mg, 13 mg, 14 mg, 15 mg, 16 mg, 17 mg, 18 mg, 19 mg, and 20 mg. The term “drug” or “therapeutic agent” as used herein is defined as a therapeutic substance, used in the prevention, diagnosis, alleviation, treatment, or cure of disease. Unless stated otherwise, “drug” and “therapeutic agent” have the same meaning.

In certain embodiments, the one or more therapeutic agents are selected from a cytostatic or anti-proliferative agent, a mTOR/PI3K dual inhibitor, a mTORC1/mTORC2 dual inhibitor, a cytotoxic agent, a 5-alpha reductase inhibitor, an alpha blocker and/or smooth muscle cell relaxer, an anti-inflammatory agent, an anti-fibrotic agent, a non-steroidal antiandrogen, and antiandrogen antineoplastic agent.

The term “cytostatic” as used herein refers to a drug that is non-toxic to cells but does mitigate cell proliferation and permit cell migration. In particular embodiments, the cytostatic or anti-proliferative agent is selected from rapamycin, sirolimus, everolimus, zotarolimus, myolimus, temsirolimus, tacrolimus, a macrolide antibiotic, a macrolide immunosuppressive drug, ridaforolimus, biolimus, novolimus, deforolimus, and structural derivatives and functional analogues thereof.

In particular embodiments, the mTOR/PI3K dual inhibitor is selected from dactolisib, BGT226, SF1126, PKI-587, and NVPBE235.

In particular embodiments, the mTORC1/mTORC2 dual inhibitor is selected from sapanisertib, AZD8055, and AZD2014.

The term “cytotoxic” as used herein refers to a drug that inhibits cell growth and proliferation such as chemotherapeutics. In particular embodiments, the cytotoxic agent is selected from paclitaxel, a taxane, protaxel, vincristine, etoposide, nocodazole, indirubin, an anthracycline derivative, daunorubicin, daunomycin, tauromustine, bofumustane, carboplatin, carmustine, cisplatin, docetaxel, cabazitaxel, doxorubicin, gemcitabine, mitomycin, procarbazine, and plicamycin.

In certain embodiments, the cytotoxic agent is an apoptotic agent. In particular embodiments, the apoptotic agent is selected from transforming growth factor beta (TGF-β) and a topoisomerase inhibitor. In more particular embodiments, the topoisomerase inhibitor is selected from 10-hydroxycamptothecin, irinotecan, and doxorubicin.

In particular embodiments, the 5-alpha reductase inhibitor is dutasteride or finasteride.

In particular embodiments, the alpha blocker and/or smooth muscle cell relaxer is selected from doxazosin, prazosin, terazosin, tamsulosin, alfuzosin, silodosin, phenoxybenzamine, vibegron, a beta 3 adregenic receptor agonist, mirabegron, and tolterodine.

In particular embodiments, the anti-inflammatory agent is selected from fluticaosone furoate, mometasone furoate, dexamethasone, prednisone, betamethasone, cortisone, hydrocortisone, methylprednisolone, a non-steroidal anti-inflammatory drug (NSAID), aspirin, ibuprofen, naproxen sodium, diclofenac, diclofenac-misoprostol, celexocib, piroxicam, indomethacin meloxicam, ketoprofen, sulindac, diflunisal, nabumetone, oxaprozin, tolmetin, salsalate, etodolac, fenoprofen, flurbiprofen, ketorolac, meclofenamate, mefenamic acid, a COX-2 inhibitor, and combinations thereof.

In particular embodiments, the non-steroidal antiandrogen is selected from aminoglutethimide, apalutamide, bicalutamide, enzalutamide, flutamide, ketoconazole, nilutamide, topilutamide and combinations thereof. In particular embodiments, the antiandrogen antineoplastic agent is abiraterone acetate.

In certain embodiments, the bioabsorbable polymer is selected from a silk-elastin like protein polymer, Pluronics F68, Pluronics F127, or a combination thereof, poly(ε-caprolactone) (PCL), a polylactide (PLA), poly(D,L-lactide) (PDLA), a poly(ortho ester), a polyanhydride, a polycarbonate, polyethylene glycol (PEG), polyethylene oxide (PEO), a polyesteramide, and combinations and block and random co-polymers thereof.

In particular embodiments, the co-polymer is selected from poly(lactide-co-glycolide) (PLGA) and PLGA-PEG-PLGA.

In more particular embodiments, the PLGA is selected from poly(D,L-lactide-co-glycolide) (50:50), poly(D-lactide-co-glycolide) (50:50), poly(L-lactide-co-glycolide) (50:50), poly(D,L-lactide-co-glycolide) (65:35), poly(D-lactide-co-glycolide) (65:35), poly(L-lactide-co-glycolide) (65:35), poly(D,L-lactide-co-glycolide) (75:25), poly(D-lactide-co-glycolide) (75:25), poly(L-lactide-co-glycolide) (75:25), poly(D,L-lactide-co-glycolide) (85:15), poly(D-lactide-co-glycolide) (85:15), poly(L-lactide-co-glycolide) (85:15), poly(D,L-lactide-co-glycolide) (10:90), poly(D-lactide-co-glycolide) (10:90), and poly(L-lactide-co-glycolide) (10:90), and mixtures thereof.

The following are examples of the polymer naming nomenclature of certain PLGA polymers disclosed herein: PLGA8515A (0.3 dl/g) means poly(lactide-co-glycolide) with a monomer ratio of 85/15, end capped with an acid group (A), and an inherent viscosity of 0.3 dl/g; and PLGA6535E (0.5 dl/g) means poly(lactide-co-glycolide) with a monomer ratio of 65/35, end capped with an ester groups (E), and an inherent viscosity of 0.5 dl/g; and PLGA5050A (0.2 dl/g) means poly(lactide-co-glycolide) with a monomer ratio of 50/50, end capped with an acid group (E), and an inherent viscosity of 0.2 dl/g; and Poly(lactide-co-glycolide) is typically poly(D,L-lactide-co-glycolide) but could also be e.g. any or a mixture of poly(D,L-lactide-co-glycolide), poly(D-lactide-co-glycolide), and poly(L-lactide-co-glycolide).

In certain embodiments, the PLGA is end-capped with an end-cap group selected from an ester, an acid, an alcohol, an amine, and a thiol.

In particular embodiments, the PLGA has one or more characteristics selected from: (i) an inherent viscosity from about 0.1 dL/g to greater than about 1.0 dL/g; (ii) a molecular weight from about 10 kDa to about 150 kDa; (iii) a structure selected from linear, branched, hyperbranched, dendritic, a star structure, and a dendrimer-like star structure, and combinations thereof.

When expressing a % of a substance in the solid implant, the % of that substance may be expressed in terms of a percent weight of the drug(s) to the overall weight of the solid implant (“% X by wt”), or to the overall volume of the solid implant (“% X by vol”). Unless stated otherwise the percent dosage % will, by default, always refer to a % by weight (wt) to the total measured solid implant. Unless stated otherwise, weights are given in grams (“g”) or milligrams (“mg”), molecular weight in kilo-Daltons (“kDa”), volume in microliters (“μL”) or milliliters (“mL”), and viscosity units are expressed as inherent viscosity (i.e., the ratio of the natural logarithm of the relative viscosity to the mass concentration of the substance, such as a polymer. The unit of inherent viscosity is deciliters per gram (dL/g). A different measure of viscosity is intrinsic viscosity, which is a measure of a solute's contribution to the total viscosity. Another viscosity is dynamic viscosity or absolute viscosity, the units of which are centimeter-gram-seconds, also known as centipoise (cP).

The solid implant may comprise 0.1-60% of a polymer composition, or more preferable 30-50% of a polymer composition. The drug to polymer weight ratio of the solid implant may be 1:100, 1:50, 1:25, 1:20, 1:10, 1:3, 1:4, 1:5, 1:2, 3:5, 1:1, 3:2, 2:1 or 5:1. The solid implant, once located at the target tissue, may release 1-10%, or 11-50% of the drug load in less than 24 hours, 24-72 hours, 3-7 days, 1-4 weeks, 1-3 months or more than 3 months. The solid implant may release 80-100% in 24-72 h, 3-7 days, 1-4 weeks, 1-3 months or more than 3 months.

In some embodiments the composition may include a bioabsorbable polymer at a concentration of 20-80%, 25-75%, 40-60% by wt. of the bioabsorbable polymer composition, 80-20%, 75-25% and 0.5%-30% by wt. drug; 1%-20% by wt. of drug, or 1%-10% by wt. of drug.

The biodegradable polymer composition is preferably chosen to substantially biodegrade in a period of about 1 to 3 months, 3 to 6 months or 6 to 12 months. In some embodiments it may be desirable to formulate the solid implant so that the drug carrier is fully biodegraded before the next treatment, e.g., 3 months, 6 months or 12 months after the prior treatment. For example, the polymer would have a ratio of glycolide to lactide of 70:30 up to 15:85 for a less hydrophobic structure (faster degradation) and/or an inherent viscosity less than about 1.0 dL/g.

A polymer composition, when forming a constituent of the solid implant, is a polymer composition that enables or achieves a desired “sustained release” of the one or more drugs to the target tissue. In some embodiments, the polymer composition enables or achieves at least 50%, or up to about 100%, or substantially all drug release between 30 and 90 days, through a combination of diffusion and degradation. In other embodiments up 100% of drug release occurs from 90 to 120 days from treatment. In some embodiments, the drug has a release rate of between 5% to 50% during the first 24 hours from injecting the composition into the prostate and the drug has a release rate of no more than 10% to 75% over the first month, 25% to 95% over the first three months, and/or 50% to 100% over the first six months.

As provided hereinabove, the solid implant has to keep drug exposure to the prostate and minimize leak or flow to other surrounding organs. In addition, the solid implant should not take up too much volume in the prostate and/or bulk up the prostate.

Further, in some embodiments, the presently disclosed implant is substantially free of a radioactive agent or radioactive material. As used herein, the phrase “substantially free of a radioactive agent or radioactive material” means that the implant comprises less than trace amounts of radioactive agent or radioactive material, which, in some embodiments, is less than about 0.1% or less of radioactive agent or radioactive material.

In other embodiments, the presently disclosed subject matter provides a method for treating benign prostatic hyperplasia (BPH) in a subject in need of treatment thereof, the method comprising administering a solid implant as disclosed hereinabove at a first prostate location of the subject.

The most common types of prostate treatment are treatments for cancerous or pre-cancerous conditions (i.e., non-malignant tumors) and enlarged prostate, more commonly known as benign prostatic hyperplasia/hypertrophy (BPH). The tissue types are very different between treatments for cancer or pre-cancerous tumors vs. BPH, which calls for different formulations of a similar drug that may be effective in treating either condition. Most notably, the volume of drug (on a per-volume of prostate basis) can be very different when treating a tumor or cancer than when treating BPH. Finally, when treating for cancer or tumor one potentially wants to have the full drug release from a carrier quickly, i.e., within the first 24 hours of injecting the composition into the patient. A slow release, in contrast, is intended to mean a full drug release at a minimum of between 1 to 3 months' time from when the composition was injected into the patient.

In some embodiments, a patient to be treated has cancer in another part of the body (other than the prostate) but is being treated for BPH according to the disclosure. In some embodiments the patient does not have cancer in another part of the body and is being treated for BPH.

Procedures for treating BPH have demonstrated varying levels of efficacy and are often accompanied by undesirable or adverse effects. For example, transurethral resection of the prostate (TURP) produces improved efficacy and improvement in urinary flow rate and symptom score (IPSS) but is invasive with significant side effects including incontinence, urgency, dysuria, acute retention, stricture, ejaculation dysfunction and sexual dysfunction. Water vapor therapy and prostatic urethral lift (PUL) have demonstrated less sexual dysfunction side effects but are limited to use in smaller BPH prostates less than 80 mL and have shown less efficacy with non-responders and higher retreatment rates compared to TURP. Furthermore, these procedures are invasive and require transurethral access and catheter placement.

Less invasive targeted drug delivery approaches to the prostate zone have been attempted by the transrectal or transperineal routes, such as pore-forming proteins and peptides in saline formulations with single dosages, but demonstrated limited efficacy versus saline placebo in randomized clinical trials. See Indian J Urol. 2008 July-September; 24 (3): 329-335. doi: 10.4103/0970-1591.42613, PMCID: PMC2684358, PMID: 19468462; Injection therapy for prostatic disease: A renaissance concept. Arash M. Saemi, Jeffrey B. Folsom, and Mark K. Plante. Additionally, alcohol or medications injected into the prostate have been ineffective or difficult to control. Alcohol single injection is very caustic and poorly controls the area of delivery. Medication injection into the prostate also has been ineffective as it is given in a single dose with poor effect. Other attempts to treat prostate using similar drugs and/or peptide drugs have been used. If injected, the injectate did not include a sustained release formulation of the drug and thus a long acting, efficacious response in the target tissue injected would not be exhibited.

While the foregoing methods may show efficacy in reducing BPH, they either require a more invasive procedure (vs. localized treatment using the delivery device as disclosed herein), more frequent treatment due to diffusion or more generalized treatment of BPH raising the possibility of adverse effects because a comparatively high dosage of the drug is needed to treat the area while accounting for leakage or diffusion of the drug to other areas (i.e., at least 2 to 3 times higher dose of the drug compared to the drug dosage of the presently disclosed solid implant). Adverse effects may include diminished urinary or sexual function. It is desired to have an effective treatment targeting only the target tissue and nowhere else (e.g., avoiding the bladder, urethra and nerves surrounding the prostate) and to perform the procedure in a less invasive manner for patient acceptance.

Access to prostatic tissue may be achieved in a transurethral or transrectal manner via an existing body orifice or through transperineal entry. It may be beneficial and less invasive to access the tissue by either transrectal or transperineal approaches. The advantages with a transrectal or transperineal approach include, but are not limited to: (1) oral and/or local anesthesia application instead of general anesthesia; (2) less trauma to the urethra tract and less resulting side effects also reducing the need for post procedure catheterization; (3) faster recovery time for the patient; (4) familiar treatment for the urologist physician similar to prostate cancer biopsy; and combinations thereof.

For access by transrectal or transperineal approach, guidance may be provided by ultrasound, x-ray, computed tomography, magnetic resonance imaging or other imaging modality. Ultrasound imaging may be beneficial given that ultrasound is routinely utilized for prostate biopsy. The transrectal and transperineal approaches closely mirrors the present prostate ultrasound and biopsy techniques familiar to urologists. Transrectal and transperineal approaches both avoid interaction with the urethra, which limits the caustic effects of urethral procedures, therefore minimizing side effects and dysuria associated with current BPH procedures.

Accordingly, in certain embodiments, the implant is administered by injection through a rectum, a perineum, or an urethra of the subject. Delivery of the presently disclosed solid implants by way of local prostate needle injection overcomes many of the drawbacks with existing procedures for treating BPH. For example, the presently disclosed solid implant can be delivered to the prostate on a consistent, repeatable basis to ensure administration of a total dosage per prostate size is substantially met, but not exceeded, by an administering health professional, to achieve the desired outcome (i.e., reduction in urethral obstruction and/or prostate volume over a period of time by a programmed, sustained release of a drug) and without adverse indications (e.g., infection, post-treatment pain or discomfort, and drug diffusion into nearby areas, such as the urethra or bladder). A prostate treatment according to the disclosure may be described as a prostate treatment having the following characteristics:
(1) MINIMAL UNCONTROLLED DRUG DIFFUSION: Upon contact with the prostate or prostate tissue, the drug or therapeutic agent comprising the solid implant remains at the target tissue and little or no drug/agent diffusion takes place to possibly affect unintended, adjacent tissue, such as the urethra, bladder, or seminal vesicles;
(2) SUSTAINED RELEASE & EFFICACY: Enabling a controlled release of a drug over time in a sufficient total dose to achieve the efficacious result of reduced prostate size; and
(3) LOW VOLUME: Delivery of the solid implant total volume to the prostate using a needle delivery device. This manner of delivery avoids causing additional acute pressure on the urethra, which may cause additional discomfort for the patients immediately after or shortly after the procedure. It also can be desirable to have a low volume injected at several discrete, nearby locations to achieve the targeted clinical outcome, as this can optimize diffusion of a hydrophobic drug relative to diffusion from a large volume injected in one location.

Accordingly, in some embodiments, the implant is administered by injection with a needle having a length from about 15 cm to about 20 cm, including about 15 cm, 16 cm, 17 cm, 18 cm, 19 cm, 20 cm, and 25 cm. In certain embodiments, the needle comprises a lumen having a diameter from about 0.02 cm to about 0.12 cm, including a diameter of about 0.02 cm, 0.03 cm, 0.04, cm, 0.05 cm, 0.06 cm, 0.07 cm, 0.08 cm, 0.09 cm, 0.10 cm, 0.11 cm, and 0.12 cm. In certain embodiments, the needle has a ratio of length divided by lumen diameter of between about 100 and 1000, including a ratio of length divided by lumen diameter of about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and 1000, or, in some embodiments, between about 230 and 340, including 230, 235, 240, 245, 250, 255, 260, 265, 270, 275, 280, 285, 290, 295, 300, 305, 310, 315, 320, 325, 330, 335, and 340.

In certain embodiments, the solid implants are delivered by a needle device that deploys each implant by a contact force. This contact force may be delivered by a solid, liquid or gas. A solid contact force may be delivered by a plunger or stylet to physically push out each solid implant from the needle to the prostate tissue. In this case the plunger can be mechanically actuated by the user such as pushing forward a stylet. The mechanical actuation can also be spring activated, switch or ratchet activated. The delivery device may include an ergonomic handle and switch, ratchet, rotatable gears, slidable and/or spring loaded button for the user to deliver the solid implant. Examples of these mechanisms were described earlier in connection with FIGS. 3, 4 and 6. A liquid or gas contact force may also be utilized to deliver the solid implant. The liquid or gas medium does not solubilize or dissolve the implant and is biocompatible. For example, sterile saline fluid may be utilized or medical grade oxygen and/or oxygen nitrous oxide mixtures. A syringe or pressure valve can be utilized to deliver the implant with liquid or gas media. The delivery device may be activated by release of a safety lock to prevent unintended delivery.

In certain embodiments, the implant is administered via a needle guided by ultrasound imaging. The imaging device, e.g., ultrasound, may be used both as a needle guide to the target tissue and for sizing the prostate. Once the prostate size is determined, the number, n, of solid implants for injection may be determined. A needle syringe according to the disclosure may hold one or a plurality of solid implants dispensable from the needle to treat the prostate in a controlled manner.

In certain embodiments, the method comprises administering up to 20 implants, including 1, 2, 3, 4, 5, 6, 7, 8, 9, 10, 11, 12, 13, 14, 15, 16, 17, 18, 19, and 20 implants. In particular embodiments, the method comprises administering between 10 and 20 implants, including about 10, 11, 12, 13, 14, 15, 16, 17, 18, 19, and 20 implants. Treatment of BPH by a needle injection in a small quantity at a plurality of locations in the target tissue offers several benefits over other methods. Among the benefits are less invasive procedures leading to greater patient acceptance and less complications during patient treatment, less frequent procedures needed, and reduced incidence of drug affecting nearby tissue leading to such outcomes as adverse consequences for urinary or sexual function.

For delivery of multiple small volume solid implants, multiple delivery devices and long slender needles may be utilized. Alternatively, the user can reload additional small volume solid implants each time. A user loading tool may be used for ease in handling the small volume implants that fits on the distal needle and guides the implants into the distal needle. In each case the plunger, liquid or gas media can be reset or further displaced for the subsequent small volume solid implant delivery. Alternatively for ease of use and minimal user contact to the sterile implant, the delivery device may be loaded with multiple small volume solid implants. Multiple small volume cylindrical implants may be pre-loaded or primed in the delivery device long slender needle and the plunger or stylet preset or marked for delivery of each implant.

In certain embodiments, the one or more therapeutics agents are eluted over a time period of between about 1 months to about 3 months, 3 months to about 6 months, including about 3 months, 4 months, 5 months, and 6 months and 6 months to about 12 months.

In certain embodiments, administration of the implant reduces pressure on the subject's urethra through tissue atrophy and prevention or reduced rate of further prostate tissue growth.

Referring once again to FIG. 6A-6C, in some embodiments, the presently disclosed method further includes moving a plunger relative to a needle to deploy the implant. In other embodiments, the method further includes rotating a wheel to deploy the implant. In yet other embodiments, a slider is coupled to a plunger, and the method further includes linearly moving the slider along a track to deploy the implant.

The treatment may be used in combination with, or in addition to treatments involving removal/ablation of tissue, and/or delivery of energy to the tissue and additionally the administering of various agents. Methods according to the disclosure may additionally, or alternatively, be administered after a treatment of BPH according to other methods.

In yet other embodiments, the presently disclosed subject matter provides a system for treating benign prostatic hyperplasia (BPH) in a subject in need of treatment thereof, the system comprising: (i) one or more preformed solid implants as described hereinabove; and (ii) an applicator or delivery system for local delivery of the one or more implants to target tissue of the subject's prostate.

The term “target tissue” as used herein is defined as prostate tissue to include the transition zone, peripheral zone and central zone of the prostate, and the prostate. As used herein, the term “transition zone” surrounds the proximal urethra and is the region of the prostate gland that grows throughout life and is responsible for the disease of benign prostatic enlargement. The “central zone” surrounds the ejaculatory duct apparatus and makes up the majority of the prostatic base.

In certain embodiments, the applicator or delivery system comprises a needle, wherein the needle has one or more characteristics selected from: (i) a length from about 15 cm to about 25 cm; (ii) a lumen having a diameter from about 0.02 cm to about 0.12 cm; and (iii) a ratio of length divided by lumen diameter of between about 200 and 400.

In particular embodiments, the system further includes an ultrasound device for locating target tissue in the prostate. In certain embodiments, the system further includes a force generating element to deploy the implants, such as a plunger.

In further embodiments, the applicator or delivery system includes: a needle with a lumen; wherein the implant is positioned in the lumen; and a plunger at least partially positioned within the lumen and movable with respect to the needle.

In another embodiment the implant and/or device needle are echogenic to make more visible during ultrasound imaging.

In certain embodiments, the system further includes a plurality of solid implants pre-loaded in the needle and a force generating plunger element to deploy each implant by the user. In particular embodiments, the plunger includes a neck with a plurality of indicia corresponding to the administration of each of the plurality of implants.

In some embodiments, the device further includes a first detent on the needle and a second detent on the neck, and wherein the first detent and the second detent provide a haptic feedback and/or an audible feedback corresponding to the administration of each of the plurality of implants.

In certain embodiments, the applicator or delivery system further includes a handle control including a housing.

In some embodiments, the system includes a rotary mechanism with at least one wheel engaged with a rack coupled to the plunger. In certain embodiments, the system further includes a detent formed between the at least one wheel and the housing.

In some embodiments, the system further includes a slider coupled to the plunger and movable along a track formed in the housing. In particular embodiments, the system further includes indicia position along the track.

In some embodiments, the system further includes a lock selectively coupled to the plunger, wherein movement of the plunger is prevented when the lock is coupled to the plunger.

In some embodiments, the system further includes a spring mechanism coupled to the plunger.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

PLGA5050 is dissolved at 10% in acetone along with 50% paclitaxel and then pipetted into anodized aluminum or silicone molds. Solvent is then allowed to evaporate at 37° C. in an oven for 30 min to 24 hours. Final implants, 6-mm long×0.6-mm diameter, each containing approximately 1 mg paclitaxel that elutes over 3 months in vivo, are removed from the mold.

### Example 2

PLGA7525 is dissolved at 20% in acetone along with 50% paclitaxel and then pipetted into anodized aluminum or silicone molds. Solvent is then allowed to evaporate at 37° C. in an oven for 30 min to 24 hours. Final implants, 6-mm long×0.6 mm diameter, each containing approximately 1 mg paclitaxel that elutes over 6 months in vivo, are removed from the mold.

### Example 3

PLGA5050 is dissolved at 10% in acetone along with 50% sirolimus and then pipetted into anodized aluminum or silicone molds. Solvent is then allowed to evaporate at 37° C. in an oven for 30 min to 24 hours. Final implants, 6-mm long×0.6-mm diameter, each containing approximately 1 mg paclitaxel that elutes over 3 months in vivo, are removed from the mold.

### Example 4

PLGA7525 is dissolved at 20% in acetone along with 50% sirolimus and then pipetted into anodized aluminum or silicone molds. Solvent is then allowed to evaporate at 37° C. in an oven for 30 min to 24 hours. Final implants, 6-mm long×0.6-mm diameter, each containing approximately 1 mg paclitaxel each that elutes over 6 months in vivo, are removed from the mold.

### Example 5

PLGA5050 is dissolved at 10% in NMP along with 50% paclitaxel and then direct injected into sterile water for injection bath using a 27G stainless steel needle to precipitate the solid implant. The implants are removed from the water bath and the solvent is allowed to evaporate at 37° C. in an oven for 30 min to 24 hours. Final implants, 6-mm long×0.6-mm diameter, each containing approximately 1 mg paclitaxel that elutes over 3 months in vivo, are removed from the mold.

### Example 6

PLGA7525 is dissolved at 10% in NMP along with 50% paclitaxel and then direct injected into sterile water for injection bath using a 27G stainless steel needle to precipitate the solid implant. The implants are removed from the water bath and the solvent is allowed to evaporate at 37° C. in an oven for 30 min to 24 hours. Final implants, 6-mm long×0.6-mm diameter, each containing approximately 1 mg paclitaxel that elutes over 6 months in vivo, are removed from the mold.

### Example 7

PLGA5050 is dissolved at 10% in NMP along with 50% sirolimus and then direct injected into sterile water for injection bath using a 27G stainless steel needle to precipitate the solid implant. The implants are removed from the water bath and the solvent is allowed to evaporate at 37° C. in an oven for 30 min to 24 hours. Final implants, 6-mm long×0.6-mm diameter, each containing approximately 1 mg paclitaxel that elutes over 3 months in vivo, are removed from the mold.

### Example 8

PLGA7525 is dissolved at 10% in NMP along with 50% sirolimus and then direct injected into sterile water for injection bath using a 27G stainless steel needle to precipitate the solid implant. The implants are removed from the water bath and the solvent is allowed to evaporate at 37° C. in an oven for 30 min to 24 hours. Final implants, 6-mm long×0.6-mm diameter, each containing approximately 1 mg paclitaxel that elutes over 6 months in vivo, are removed from the mold.

### Example 9

PLGA5050 is dissolved at 25% in acetone along with 25% paclitaxel and then pipetted into anodized aluminum or silicone molds. Solvent is then allowed to evaporate at 37° C. in an oven for 120 min. Final implants, 6-mm long×0.6-mm diameter, each containing approximately 3 mg paclitaxel that elutes over 3 months in vivo, are removed from the mold.

### Example 10

PLGA5050 is dissolved at 20% in acetonitrile along with 20% paclitaxel and then pipetted into anodized aluminum or silicone molds. Solvent is then allowed to evaporate at 25° C. in an oven for 24 h. Final implants, 6-mm long×0.6-mm diameter, each containing approximately 3 mg paclitaxel that elutes over 3 months in vivo, are removed from the mold.

### Example 11

PLGA5050 is dissolved at 20% in acetonitrile along with 20% doxorubicin and then pipetted into anodized aluminum or silicone molds. Solvent is then allowed to evaporate at 25° C. in an oven for 24 h. Final implants, 6-mm long×0.6-mm diameter, each containing approximately 3 mg doxorubicin 1 that elutes over 3 months in vivo, are removed from the mold.

### Example 12

PLGA5050 is dissolved at 20% in dichloromethane along with 20% paclitaxel and then pipetted into anodized aluminum or silicone molds. Solvent is then allowed to evaporate at 25° C. in an oven for 24 h. Final implants, 6-mm long×0.6-mm diameter, each containing approximately 3 mg paclitaxel that elutes over 3 months in vivo, are removed from the mold.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

U.S. Pat. No. 11,602,516 for Treating benign prostatic hyperplasia, to Trollsas et al., issued Mar. 14, 2023.

U.S. Patent Application Publication No. 2022/0031606A1 for System and method for prostrate treatment, to Trollsas et al., published Feb. 3, 2022.

Although the foregoing subject matter has been described in some detail by way of illustration and example for purposes of clarity of understanding, it will be understood by those skilled in the art that certain changes and modifications can be practiced within the scope of the appended claims.

### CLAUSES

Clause 1. A preformed injectable solid implant for treating benign prostatic hyperplasia (BPH), the implant comprising one or more therapeutic agents in a bioabsorbable polymer, wherein the implant has a shape selected from a rod, spheroid, grain, and pellet, and a size in a first dimension (a) between about 0.3 mm and about 6 mm and a size in a second dimension (b) between about 0.2 mm and about 1.5 mm, provided that the size of the first dimension (a) is greater than the size in the second dimension (b).

Clause 2. The implant of Clause 1, wherein the size of the first dimension (a) and the size of the second dimension (b) is selected from about 6 mm×1.5 mm, 4 mm×1.5 mm, 3 mm×1.5 mm, 2 mm×1.5 mm, 6 mm×1 mm, 4 mm×1 mm, 3 mm×1 mm, 2 mm×1 mm, 1.5 mm×1 mm, 6 mm×0.5 mm, 4 mm×0.5 mm, 3 mm×0.5 mm, 2 mm×0.5 mm, 1.5 mm×0.5 mm, 1 mm×0.5 mm, 6 mm×0.4 mm, 4 mm×0.4 mm, 3 mm×0.4 mm, 2 mm×0.4 mm, 1.5 mm×0.4 mm, 1 mm×0.4 mm, 6 mm×0.3 mm, 4 mm×0.3 mm, 3 mm×0.3 mm, 2 mm×0.3 mm, 1.5 mm×0.3 mm, 1 mm×0.3 mm, 0.5 mm×0.3 mm, 6 mm×0.2 mm, 4 mm×0.2 mm, 3 mm×0.2 mm, 2 mm×0.2 mm, 1.5 mm×0.2 mm, 1 mm×0.2 mm, 0.5 mm×0.2 mm, and 0.3 mm×0.2 mm.

Clause 3. The implant of Clause 1 or Clause 2, wherein the implant has a weight having a range from about 1 mg to about 6 mg.

Clause 4. The implant of any one of Clauses 1-3, wherein the implant has a therapeutic agent to polymer ratio by weight is selected from about 1 to 10, 1 to 5, 1 to 4, 1 to 3, 1 to 2, 6 to 10, 1 to 1 and 3 to 2.

Clause 5. The implant of any one of Clauses 1-4, wherein the one or more therapeutic agents have a dose ranging from about 0.1 mg to about 6 mg.

Clause 6. The implant of Clause 5, comprising a plurality of implants having a total dose of one or more therapeutic agents ranging from about 2 mg to 71 mg.

Clause 7. The implant of any one of Clauses 1-6, wherein the one or more therapeutic agents are selected from a cytostatic or anti-proliferative agent, a mTOR/PI3K dual inhibitor, a mTORC1/mTORC2 dual inhibitor, a cytotoxic agent, a 5-alpha reductase inhibitor, an alpha blocker and/or smooth muscle cell relaxer, an anti-fibrotic agent, a non-steroidal antiandrogen, and antiandrogen antineoplastic agent.

Clause 8. The implant of Clause 7, wherein the cytostatic or anti-proliferative agent is selected from rapamycin, sirolimus, everolimus, zotarolimus, myolimus, temsirolimus, tacrolimus, a macrolide antibiotic, a macrolide immunosuppressive drug, ridaforolimus, biolimus, novolimus, deforolimus, and structural derivatives and functional analogues thereof.

Clause 9. The implant of Clause 7, wherein the cytotoxic agent is selected from paclitaxel, a taxane, protaxel, vincristine, etoposide, nocodazole, indirubin, an anthracycline derivative, daunorubicin, daunomycin, tauromustine, bofumustane, carboplatin, carmustine, cisplatin, docetaxel, cabazitaxel, doxorubicin, gemcitabine, mitomycin, procarbazine, and plicamycin.

Clause 10. The implant of Clause 7, wherein the cytotoxic agent is an apoptotic agent.

Clause 11. The implant of Clause 7, wherein the 5-alpha reductase inhibitor is dutasteride or finasteride.

Clause 12. The implant of Clause 7, wherein the alpha blocker and/or smooth muscle cell relaxer is selected from doxazosin, prazosin, terazosin, tamsulosin, alfuzosin, silodosin, phenoxybenzamine, vibegron, a beta 3 adregenic receptor agonist, mirabegron, and tolterodine.

Clause 13. The implant of Clause 7, wherein the non-steroidal antiandrogen is enzalutamide.

Clause 14. The implant of any one of Clauses 1-13, wherein the bioabsorbable polymer is selected from a silk-elastin like protein polymer, Pluronics F68, Pluronics F127, or a combination thereof, poly(ε-caprolactone) (PCL), a polylactide (PLA), poly(D,L-lactide) (PDLA), a poly(ortho ester), a polyanhydride, a polycarbonate, polyethylene glycol (PEG), polyethylene oxide (PEO), a polyesteramide, and any combinations of block or random co-polymers thereof.

Clause 15. The implant of Clause 14, wherein the co-polymer is selected from poly(lactide-co-glycolide) (PLGA) or PLGA-PEG-PLGA.

Clause 16. The implant of Clause 15, wherein the PLGA is selected from poly(D,L-lactide-co-glycolide) (50:50), poly(D-lactide-co-glycolide) (50:50), poly(L-lactide-co-glycolide) (50:50), poly(D,L-lactide-co-glycolide) (65:35), poly(D-lactide-co-glycolide) (65:35), poly(L-lactide-co-glycolide) (65:35), poly(D,L-lactide-co-glycolide) (75:25), poly(D-lactide-co-glycolide) (75:25), poly(L-lactide-co-glycolide) (75:25), poly(D,L-lactide-co-glycolide) (85:15), poly(D-lactide-co-glycolide) (85:15), poly(L-lactide-co-glycolide) (85:15), poly(D,L-lactide-co-glycolide) (10:90), poly(D-lactide-co-glycolide) (10:90), and poly(L-lactide-co-glycolide) (10:90), and mixtures thereof.

Clause 17. The implant of Clause 16, wherein the PLGA has one or more characteristics selected from:
(i) an inherent viscosity from about 0.1 dL/g to greater than about 1.0 dL/g;
(ii) an absolute molecular weight from about 10 kDa to about 150 kDa;
(iii) a structure selected from linear, branched, hyperbranched, dendritic, a star structure, and a dendrimer-like star structure; and combinations thereof.

Clause 18. A method for treating Benign Prostatic Hyperplasia (BPH) in a subject in need of treatment thereof, the method comprising administering an implant of any one of Clauses 1-17 at a first prostate location of the subject.

Clause 19. The method of Clause 18, wherein the implant is administered through a rectum, a perineum, or an urethra of the subject.

Clause 20. The method of Clause 19, wherein the implant is administered with a needle having a length from about 15 cm to about 25 cm.

Clause 21. The method of Clause 20, wherein the delivery needle or lumen comprises a lumen having a diameter from about 0.02 cm to about 0.12 cm.

Clause 22. The method of Clause 20, wherein the delivery needle or lumen has a ratio of length divided by lumen diameter of between about 200 and 400.

Clause 23. The method of any one of Clauses 18-22, wherein the first prostate location comprises a transition zone of the subject's prostate.

Clause 24. The method of any one of Clauses 18-23, wherein the implant is administered via a delivery needle or lumen guided by ultrasound imaging.

Clause 25. The method of any one of Clauses 18-24, wherein administration of the implant reduces pressure on the subject's urethra through tissue atrophy.

Clause 26. The method of any one of Clauses 18-25, comprising administering up to 20 implants.

Clause 27. The method of Clause 26, comprising administering between 2 and 20 implants.

Clause 28. The method of any one of Clauses 18-27, wherein the one or more therapeutics agents are eluted over a time period of between about 3 months to about 6 months or 6 months to 24 months.

Clause 29. A system for treating benign prostatic hyperplasia (BPH) tissue in a subject in need of treatment thereof, the system comprising:
(i) a plurality of preformed injectable solid implants for treating benign prostatic hyperplasia (BPH), wherein an implant comprises one or more therapeutic agents in a bioabsorbable polymer, a shape selected from a rod, spheroid, grain, and pellet, and a size in a first dimension (a) between about 0.3 mm and about 6 mm and a size in a second dimension (b) between about 0.2 mm and about 1.5 mm, provided that the size of the first dimension (a) is greater than the size in the second dimension (b); and
(ii) an applicator or delivery system for local delivery of the implants to a target tissue of the subject's prostate.

Clause 30. The system of Clause 29, the applicator or delivery system further comprising:
a first lumen of the applicator or delivery system holding at least some of the plurality of implants, and a second lumen including a needle tip,
wherein the second lumen has a size between 2% and 10%, 5% and 15%, 5% and 10%, or 6% to 8% greater than the second dimension, and
wherein the applicator or delivery system enables an operator of the applicator or delivery system to release only a first implant of the plurality of implants from the needle tip when an actuator of the applicator or delivery system applies pressure to the first implant, the first implant being disposed within the second lumen when the pressure is applied, whereby the second lumen size enables the first implant to travel along the first lumen without rotating or displacing off-axis within the second lumen when pressure is applied.

Clause 31. The system of Clause 30, wherein the applicator or delivery system includes the first implant and a second implant disposed within the needle lumen, such that the first implant may be deposited, followed by the second implant at a same or different location in the prostate.

Clause 32. The system of Clause 30, wherein the first lumen comprises the second lumen.

Clause 33. The system of any one of Clauses 29-32, wherein the size of the first dimension (a) and the size of the second dimension (b) is selected from about 6 mm×1.5 mm, 4 mm×1.5 mm, 3 mm×1.5 mm, 2 mm×1.5 mm, 6 mm×1 mm, 4 mm×1 mm, 3 mm×1 mm, 2 mm×1 mm, 1.5 mm×1 mm, 6 mm×0.5 mm, 4 mm×0.5 mm, 3 mm×0.5 mm, 2 mm×0.5 mm, 1.5 mm×0.5 mm, 1 mm×0.5 mm, 6 mm×0.4 mm, 4 mm×0.4 mm, 3 mm×0.4 mm, 2 mm×0.4 mm, 1.5 mm×0.4 mm, 1 mm×0.4 mm, 6 mm×0.3 mm, 4 mm×0.3 mm, 3 mm×0.3 mm, 2 mm×0.3 mm, 1.5 mm×0.3 mm, 1 mm×0.3 mm, 0.5 mm×0.3 mm, 6 mm×0.2 mm, 4 mm×0.2 mm, 3 mm×0.2 mm, 2 mm×0.2 mm, 1.5 mm×0.2 mm, 1 mm×0.2 mm, 0.5 mm×0.2 mm, and 0.3 mm×0.2 mm.

Clause 34. The system of any one of Clauses 29-33, wherein the applicator or delivery system comprises a needle or delivery lumen, wherein the needle or delivery lumen has one or more characteristics selected from:
(i) a length from about 15 cm to about 25 cm;
(ii) a lumen having a diameter from about 0.06 cm to about 0.12 cm; and
(iii) a ratio of length divided by lumen diameter of between about 100 and 400.

Clause 35. The system of any one of Clauses 29-34, wherein the system further includes an ultrasound device for locating target tissue in the prostate.

Clause 36. The system of any one of Clauses 29-35, wherein the applicator or delivery system includes:
a needle with a lumen; wherein the implant is positioned in the lumen; and
a plunger at least partially positioned within the lumen and movable with respect to the needle.

Clause 37. The system of Clause 36, further including a plurality of solid implants pre-loaded in the needle or delivery lumen and a force generating plunger element to deploy each implant by the user.

Clause 38. The system of Clause 37, wherein the plunger includes a neck with a plurality of indicia corresponding to the administration of each of the plurality of implants.

Clause 39. The system of Clause 37, wherein the device further includes a first detent on the needle and a second detent on the neck, and wherein the first detent and the second detent provide a haptic feedback and/or an audible feedback corresponding to the administration of each of the plurality of implants.

Clause 40. The system of Clause 37, wherein the applicator or delivery system further includes a handle control including a housing.

Clause 41. The system of Clause 40, further including a rotary mechanism with at least one wheel engaged with a rack coupled to the plunger.

Clause 42. The system of Clause 41, further including a detent formed between the at least one wheel and the housing.

Clause 43. The system of Clause 40, further including a slider coupled to the plunger and movable along a track formed in the housing.

Clause 44. The system of Clause 43, further including indicia position along the track.

Clause 45. The system of Clause 43, further including a lock selectively coupled to the plunger, wherein movement of the plunger is prevented when the lock is coupled to the plunger.

Clause 46. The system of Clause 43, further including a spring mechanism coupled to the plunger.

Clause 47. An implant of any one of Clauses 1-17 for use in treating Benign Prostatic Hyperplasia (BPH) in a subject.

Clause 48. The implant for use of Clause 47, wherein the implant is administered through a rectum, a perineum, or an urethra of the subject.

Clause 49. The implant for use of Clause 48, wherein the implant is administered with a needle having a length from about 15 cm to about 25 cm.

Clause 50. The implant for use of Clause 49, wherein the delivery needle or lumen comprises a lumen having a diameter from about 0.02 cm to about 0.12 cm.

Clause 51. The implant for use of Clause 49, wherein the delivery needle or lumen has a ratio of length divided by lumen diameter of between about 200 and 400.

Clause 52. The implant for use of any one of Clauses 47-51, wherein the first prostate location comprises a transition zone of the subject's prostate.

Clause 53. The implant for use of any one of Clauses 47-52, wherein the implant is administered via a delivery needle or lumen guided by ultrasound imaging.

Clause 54. The implant for use of any one of Clauses 47-53, wherein administration of the implant reduces pressure on the subject's urethra through tissue atrophy.

Clause 55. The implant for use of any one of Clauses 47-54, comprising administering up to 20 implants.

Clause 56. The implant for use of Clause 55, comprising administering between 2 and 20 implants.

Clause 57. The implant for use of any one of Clauses 47-56, wherein the one or more therapeutics agents are eluted over a time period of between about 3 months to about 6 months or 6 months to 24 months.

Clause 58. An implant of any one of Clauses 1-17 for use in preparing a medicament for treating Benign Prostatic Hyperplasia (BPH) in a subject.

That which is claimed:

1. A system for treating benign prostatic hyperplasia (BPH) tissue in a subject in need of treatment thereof, the system comprising:
(i) a plurality of preformed injectable solid implants for treating benign prostatic hyperplasia (BPH), wherein an implant comprises one or more therapeutic agents in a bioabsorbable polymer, a shape selected from a rod, spheroid, grain, and pellet, and a size in a first dimension (a) between about 0.3 mm and about 6 mm and a size in a second dimension (b) between about 0.2 mm and about 1.5 mm, provided that the size of the first dimension (a) is greater than the size in the second dimension (b); and
(ii) an applicator or delivery system for local delivery of the implants to a target tissue of the subject's prostate.

2. The system of claim 1, the applicator or delivery system further comprising:
a first lumen of the applicator or delivery system holding at least some of the plurality of implants, and a second lumen including a needle tip,
wherein the second lumen has a size between 2% and 10%, 5% and 15%, 5% and 10%, or 6% to 8% greater than the second dimension, and
wherein the applicator or delivery system enables an operator of the applicator or delivery system to release only a first implant of the plurality of implants from the needle tip when an actuator of the applicator or delivery system applies pressure to the first implant, the first implant being disposed within the second lumen when the pressure is applied, whereby the second lumen size enables the first implant to travel along the first lumen without rotating or displacing off-axis within the second lumen when pressure is applied.

3. The system of claim 2, wherein the applicator or delivery system includes the first implant and a second implant disposed within the needle lumen, such that the first implant may be deposited, followed by the second implant at a same or different location in the prostate.

4. The system of claim 2, wherein the first lumen comprises the second lumen.

5. The system of claim 1, wherein the size of the first dimension (a) and the size of the second dimension (b) is selected from about 6 mm×1.5 mm, 4 mm×1.5 mm, 3 mm×1.5 mm, 2 mm×1.5 mm, 6 mm×1 mm, 4 mm×1 mm, 3 mm×1 mm, 2 mm×1 mm, 1.5 mm×1 mm, 6 mm×0.5 mm, 4 mm×0.5 mm, 3 mm×0.5 mm, 2 mm×0.5 mm, 1.5 mm×0.5 mm, 1 mm×0.5 mm, 6 mm×0.4 mm, 4 mm×0.4 mm, 3 mm×0.4 mm, 2 mm×0.4 mm, 1.5 mm×0.4 mm, 1 mm×0.4 mm, 6 mm×0.3 mm, 4 mm×0.3 mm, 3 mm×0.3 mm, 2 mm×0.3 mm, 1.5 mm×0.3 mm, 1 mm×0.3 mm, 0.5 mm×0.3 mm, 6 mm×0.2 mm, 4 mm×0.2 mm, 3 mm×0.2 mm, 2 mm×0.2 mm, 1.5 mm×0.2 mm, 1 mm×0.2 mm, 0.5 mm×0.2 mm, and 0.3 mm×0.2 mm.

6. The system of claim 1, wherein the applicator or delivery system comprises a needle or delivery lumen, wherein the needle or delivery lumen has one or more characteristics selected from:
(i) a length from about 15 cm to about 25 cm;
(ii) a lumen having a diameter from about 0.06 cm to about 0.12 cm; and
(iii) a ratio of length divided by lumen diameter of between about 100 and 400.

7. The system of claim 1, wherein the system further includes an ultrasound device for locating target tissue in the prostate.

8. The system of claim 1, wherein the applicator or delivery system includes:
a needle with a lumen; wherein the implant is positioned in the lumen; and
a plunger at least partially positioned within the lumen and movable with respect to the needle.

9. The system of claim 8, further including a plurality of solid implants pre-loaded in the needle or delivery lumen and a force generating plunger element to deploy each implant by the user.

10. The system of claim 9, wherein the plunger includes a neck with a plurality of indicia corresponding to the administration of each of the plurality of implants.

11. The system of claim 9, wherein the device further includes a first detent on the needle and a second detent on the neck, and wherein the first detent and the second detent provide a haptic feedback and/or an audible feedback corresponding to the administration of each of the plurality of implants.

12. The system of claim 9, wherein the applicator or delivery system further includes a handle control including a housing.

13. The system of claim 12, further including a rotary mechanism with at least one wheel engaged with a rack coupled to the plunger.

14. The system of claim 13, further including a detent formed between the at least one wheel and the housing.

15. The system of claim 12, further including a slider coupled to the plunger and movable along a track formed in the housing.

16. The system of claim 15, further including indicia position along the track.

17. The system of claim 15, further including a lock selectively coupled to the plunger, wherein movement of the plunger is prevented when the lock is coupled to the plunger.

18. The system of claim 15, further including a spring mechanism coupled to the plunger.
